(19) United States
(12) Patent Application Publication
(10) Pub. No.: US 2007/0256147 A1
(43) Pub. Date: Nov. 1, 2007

(54) Combinatorial marking of cells and cell structures with reconstituted fluorescent proteins

(76) Inventors: Martin Chalfie, New York, NY (US); Charles Ma, Palo Alto, CA (US); Shifang Zhang, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/633,121

(22) Filed: Dec. 1, 2006

Related U.S. Application Data
(63) Continuation of application No. PCT/US05/19717, filed on Jun. 2, 2005.
(60) Provisional application No. 60/576,487, filed on Jun. 3, 2004.

(51) Int. Cl. A01K 67/027; C07H 21/04; C12N 5/06; C12Q 1/68

(52) U.S. Cl. 800/13; 435/252.3; 435/320.1; 435/325; 435/440; 435/6; 530/350; 536/23.1; 800/295

(57) ABSTRACT

The present invention relates to the use of split fluorescent proteins to determine whether promoters are coordinately active, whereby the transcriptional expression of incomplete portions of a fluorescent protein is controlled by different promoters and coordinate (not necessarily contemporaneous) promoter activity results in the reconstitution of a fluorescent protein. The present invention, in non-limiting embodiments, may be used to selectively label cells and cell structures in vivo and to demonstrate changes in promoter activity (for example, in developmental biology and drug discovery applications).

### CROSS-REFERENCE TO RELATED APPLICATIONS

The present application is a continuation of International Patent Application No. PCT/US2005/019717 filed Jun. 2, 2005 and published in English on Dec. 15, 2005 as International Publication No. WO 2005/118790; which claims priority benefit from U.S. Provisional Patent Application Ser. No. 60/576,487, filed on Jun. 3, 2004, now abandoned, the contents of both of which are hereby incorporated by reference.

### FEDERALLY FUNDED GRANT SUPPORT

The subject matter of this application was developed at least in part under National Institutes of Health Grant GM 30997 so that the United States Government has certain rights herein.

### INTRODUCTION

The present invention relates to the use of split fluorescent proteins to determine whether or not promoters are coordinately active, whereby the transcriptional expression of incomplete portions of a fluorescent protein is controlled by different promoters and coordinate (not necessarily contemporaneous) promoter activity results in the reconstitution of a fluorescent protein. The present invention, in non-limiting embodiments, may be used to selectively label cells and cellular structures in vivo and to demonstrate changes in promoter activity (for example, in developmental biology and drug discovery applications).

### BACKGROUND OF THE INVENTION

Green fluorescent protein (“GFP”) is the source of fluorescent light emission in the jellyfish Aequorea victoria. More than a decade ago it was discovered that GFP could be used as a biological marker that could be used to visualize cellular events, in real time,—in vivo (Chalfie et al., 1994, Science 263: 802). Since then, GFP has become an important tool in many areas of biology and in many model systems. GFP has been used successfully as a reporter of promoter activity. Importantly, it has been found to maintain its fluorescent capabilities when fused to another protein, and as such, has been a valuable marker for protein localization in numerous organisms across evolutionary boundaries, including bacteria and other prokaryotes, fungi, plants, insects and other invertebrates, and mammals (for reviews see Prasher, 1995, Trends Genet. 11:320-323; Simon, 1996, Nat. Biotechnol. 14:1221; Tsien, 1998, Annu. Rev. Biochem. 67:509-544; Zacharias et al., 2000, Curr. Opin. Neurobiol. 10:416-421; Matz et al., 2002, Bioessays 24:953-959, Zhang et al., 2002, Nat. Rev. Mol. Cell Biol. 3:906-918; Zimmer, 2002, Chem. Rev. 102:759-781; and Miyawaki, 2003, Dev. Cell 4:295-305). For example, GFP has been used in the nematode worm Caenorhabditis elegans to label cells for electrophysiology (Goodman et al., 1998, Neuron 20: 763), genetic screens (Du and Chalfie, 2001, Genetics 158: 197), and cell isolation (Zhang et al., 2002, Nature 418: 331) in addition to characterizing gene expression and protein localization.

GFP has enjoyed so much success as a biological marker that scientists have been motivated to develop other fluorescent proteins that address particular research needs (Zhang et al., 2002, Nat. Rev. Mol. Cell. Biol. 3:906-918). For example, GFP variants having altered excitation and emission wavelengths have been developed in order to simultaneously study multiple processes in a cell or organism, whereby GFP could be used to study one process, and a different “color” of fluorescent protein, such as a yellow fluorescent protein (“YFP”), cyan fluorescent protein (“CFP”), red fluorescent protein (“RFP”) or blue fluorescent protein (“BFP”) could be used concurrently to visualize another process (Sawano et al., 2000, Nucl. Acids Res. 28:E78; Griesbeck et al., 2001, J. Biol. Chem. 276:29188-29194; Nagai et al., 2002, Nature Biotechnol. 20:87-90; Scholz et al., 2000, Eur. J. Biochem. 267:1565-1570). Marine coelenterates have proven to be a fruitful source of new fluorescent proteins, and it has been reported that 30 distinct fluorescent proteins have been cloned from coelenterates such as Renilla mulleri, Heteractis crispa, Entacmaea quadricolor, Discosoma and Trachyphyllia geoffroyi (Zhang et al., 2002, Nat. Rev. Mol. Cell. Biol. 3:906-918; Ando et al., 2002, Proc. Natl. Acad. Sci. U.S.A. 99:12651-12656; Labas et al., 2002, Proc. Natl. Acad. Sci. U.S.A. 99: 4256-4261; Matz et al., 2002, Bioessays 24:953-959; Peele et al., 2001, J. Protein Chem. 20:507-519; Wiedenmann et al., 2002, Proc. Natl. Acad. Sci. U.S.A. 99:11646-11651).

There has also been a research initiative to develop tools for studying molecular interactions. Among the first such tools to be invented is the yeast two-hybrid system (Fields and Song, 1989, Nature 340:245-246), in which the interaction between two proteins, each linked to complementary domains of a transcriptional activator, results in reconstitution of the transcriptional activator and the expression of a reporter gene.

The utility of fluorescent proteins in other contexts motivated their use as markers of protein-protein interactions. For example, fluorescent proteins fused to target proteins can mark interaction between their fusion partners by Fluorescence Resonance Energy Transfer (“FRET”) a quantum mechanical phenomenon that occurs when two fluorescent molecules (a “donor” and an “acceptor”) are in proximity to one another (Zhang et al., supra, at p. 915; Tsien and Miyawaki, 1998, Science 280:1954-1955; Philipps et al., 2003, J. Mol. Biol. 327:239-249). Where the emission spectrum of the donor overlaps the excitation spectrum of the acceptor, and where the donor and acceptor are sufficiently close together (usually within 80 angstroms), energy is transferred between the pair, the donor emission is quenched and acceptor emission is increased. As the protein targets, fused to donor and acceptor fluorescent proteins, form interacting pairs, a change in the characteristics of the emitted fluorescence is observed.

More recently, fluorescent proteins have been used to detect protein interactions not by FRET, but by complementation, whereby non-fluorescent complementary portions of a fluorescent protein are fused to target proteins and the interaction between target proteins is marked by a reconstitution of fluorescence. In the late 1990's several investigators (Abedi et al., 1998, Nucleic Acids Res. 26: 623; Doi and Yanagawa, 1999, FEBS Lett. 453: 305; Baird et al., 1999, Proc. Natl. Acad. Sci. U.S.A. 96: 11241) demonstrated that the primary amino acid sequence of GFP could be interrupted at several positions by intervening coding sequences and still yield a fluorescent product. Applying this principle to detection of protein-protein interactions, Ghosh et al., 2000, J. Am. Chem. Soc. 122:5658-5659 (see also U.S. Patent Application Publication No. 2002/0146701) disclose the reconstitution of fluorescent activity upon non-covalent association between N-terminal and C-terminal portions of GFP, each fused to an antiparallel leucine zipper domain. In particular, they showed that polypeptides GFP(1-157) and GFP(158-238), which they named NGFP and CGFP, respectively, yielded a fluorescent product in vitro or when coexpressed in bacteria when linked to sequences (NZ and CZ) that could form an antiparallel leucine zipper. They designated their constructs NZGFP (NGFP+6 amino acid linker+NZ) and CZGFP (CZ+4 amino acid linker+CGFP). The Ghosh et al. results provided a proof of principle that production of fluorescence from partial GFP polypeptides joined, via their leucine zippers, to form a reconstituted GFP (hereafter, “RecGFP”), could be used to monitor protein-protein interactions.

Nagai et al. (Nagai et al., 2001, Proc. Natl. Acad. Sci. U.S.A. 98: 3197) developed another application involving the reconstitution of a fluorescent protein. Specifically, Nagai et al. demonstrated that circularly permuted GFP (in which the amino and carboxy terminal portions are interchanged and rejoined by a short spacer molecule) could be split, with one non-fluorescent half bound to calmodulin and the other bound to M13. The resulting construct reversibly produced fluorescence upon addition of calcium. These workers remarked, however, that the use of these peptides was compromised in HeLa cells because of competition by endogenous proteins.

Umezawa et al., (U.S. Patent Application Publication No. 2003/0003506; Ozawa et al. 2000, Anal. Chem. 72:5151-5157; Ozawa et al. 2001, Anal. Chem. 73:5866-5874) reconstitute fluorescent GFP by genetically fusing split VDE inteins to split GFP. U.S. Patent Application Publication No. 2003/0003506 and Ozawa et al., 2001 supra disclose a split GFP system for detecting interacting proteins in which the N-terminal half of an intein and a C-terminal half of the intein are linked, respectively, at one end to N- and C- terminal halves of split GFP. At the other ends of the intein halves are interacting proteins, A and B. When A and B interact, splicing between the inteins results, the two GFP partial polypeptides are covalently linked and severed from the other proteins, and fluorescent RecGFP is formed.

Hu and Kerppola, 2003, Nature Biotechnol. 21:539-545 (see also Hu et al., 2002, Molecular Cell 9:789-798) extend the concept of reconstituting split fluorescent proteins via protein interactions to utilize split fluorescent proteins of different colors to visualize multiple protein interactions. They used the reconstitution of fluorescent proteins (a process they refer to as “Bimolecular Fluorescence Complementation” (“BiFC”)) “to compare the dimerization selectivity and subcellular sites of interactions among basic region leucine zipper family proteins” (such as Fos and Jun).

Each of the foregoing references relate to the use of split fluorescent proteins, and their capability to form fluorescent “RecFPs,” as means for detecting and studying protein interactions. In contrast, the present invention utilizes RecFPs as markers of coordinate promoter activity. An advantage of GFP and similar fluorescent proteins is that they are genetically encoded and can be expressed in living cells and organisms from different promoters. The specificity of this expression, however, is limited by the specificity of available promoters. Often cell specificity arises from the combinatorial action of multiple regulators, and individual cell types cannot be labeled using a single regulatory element. The present invention uses RecFPs as markers of the combinatorial action of promoters driving the expression of their split fluorescent protein constituents.

### SUMMARY OF THE INVENTION

The present invention relates to the use of split fluorescent proteins as markers of coordinate promoter activity. It is based on the discovery that placing complementary portions of a fluorescent protein under the transcriptional control of two promoters that are both expressed only in a single cell type resulted in a reconstitution of fluorescent protein only in that cell type, and could also be used to label subcellular compartments in specific sets of cells.

The present invention provides an advantage over the use of intact fluorescent proteins because the activity of a given promoter is typically not sufficiently restricted, either to a single cell type, cell family or temporal context. Requiring the activity of two or more promoters to reconstitute a fluorescent protein imparts greater specificity. Furthermore, in specific non-limiting embodiments of the invention, it permits the labeling of cells and cell components that might not otherwise be labeled.

The present invention further provides a method of generating new fluorescent proteins with desirable properties, in which various complementary split fluorescent proteins carrying different sequence mutations can be used to produce RecFPs having new combinations of mutations.

Accordingly, the present invention provides for split fluorescent proteins (hereafter, “SFPs”), reconstituted fluorescent proteins (hereafter, “RecFPs”), variant FPs, nucleic acids encoding SFPs and variant FPs, vector molecules, host cells and host organisms, and kits containing the same. It further provides for methods of using SFPs and their RecFP products to demonstrate coordinate promoter activity, for example for the purpose of labeling cells and/or cellular structures, the analysis of temporal patterns of gene expression, and the identification of compounds that modulate promoter activity.

### BRIEF DESCRIPTION OF THE FIGURES

FIG. 1A-D. Reconstituted GFP (“RecGFP”) formed from split GFPs expressed from several promoters. (A) Expression of split GFP from the P_{mec-18} promoter in the six touch receptor neurons. (B) Expression of split GFP from the heat shock promoter P_{hsp16.2} throughout the animal. (C-D) Comparison of fluorescence from GFP (C) and split GFP (D) from the unc-4 promoter at various times. For P_{unc-4}gfp 6.9±0.2 cells (mean±SEM, N=50 for all), 15.4±0.3 cells, and 17.0±0.3 cells fluoresced at<2, 20, and 40 hr after hatching, respectively. For P_{unc-4}nzgfp and P_{unc-4}czgfp the equivalent values are 6.4±0.2, 5.4±0.2, and 0.4±0.1.

FIG. 2. Reconstitution of fluorescence using split fluorescent proteins with different emission spectra. The various CZ and NZ constructs are indicated to the left of the figure. All constructs were expressed from the mec-18 promoter. Fluorescence using the YFP and CFP filter sets is shown. Images from both channels were processed identically. Note that some of the images appear cyan optically, but green photographically when using the CFP filter set.

FIG. 3A-C. Use of RecGFP to identify cells coexpressing two genes, where the promoter of each gene drives expression of a split GFP linked to a leucine zipper, and the split GFPs are complementary. (A) P_{unc-24}gfp is expressed in many adult cells. (B) P_{unc-24}nzgfp and P_{mec-2}czgfp are coexpressed only in six touch receptor neurons. (C) P_{mec-3}nzgfp and P_{egl-}44czgfp are coexpressed only in the two FLP neurons.

FIG. 4A-C. Use of split GFP expressed from P_{unc-4}nzgfp and P_{acr-5}czgfp to form RecGFP and thereby characterize changes in cell fate. (A) Wild type animal, only the three SAB neurons (bar) and the PDA neuron (arrow) fluoresce; no fluorescence is seen in the ventral cord. (B) unc-4(e120) and (C) unc-37(e262) mutant-bearing animals have fluorescent cells in the ventral cord (VA motor neurons, triangles). Note that the more posterior of the SAB neurons (SABD, to the right in the figure) and the PDA cell are more intensely fluorescent in the mutant animals. The PDA process in the dorsal cord is seen in the mutants (unlabeled arrow) but not in wild type. All animals are L2-L3 larvae.

FIG. 5A-D. Use of RecGFP to characterize gene expression. (A) Pₛₜₒ₋₆gfp is expressed in many cells in the head, ventral cord, and tail of an adult. Ventral cord fluorescence is found from (B) P_{unc-4}nzgfp and Pₛₜₒ₋₆czgfp (C) P_{acr-5}nzgfp and Pₛₜₒ₋₆czgfp, but not (D) P_{unc-47}nzgfp and Pₛₜₒ₋₆czgfp in adults.

FIG. 6A-C. RecGFP can be used to label subcellular components in specific sets of cells. (A) P_{acr-5}nzgfp and Pₛₜₒ₋₆czgfp label cell bodies and processes of the B motor neurons in ventral cord. Presynaptic regions (B) and nuclei (C) are labeled in these cells using P_{acr-5}nzgfp and Pₛₜₒ₋₆snb-1::czgfp and Pₛₜₒ₋₆3Xnls::czgfp, respectively.

### DETAILED DESCRIPTION OF THE INVENTION

For purposes of clarity of description, and not by way of limitation, the detailed description is divided into the following subsections:
(i) split fluorescent proteins;
(ii) nucleic acids encoding split fluorescent protein-constructs;
(iii) host cells and organisms containing split fluorescent protein-constructs;
(iv) use of the invention to demonstrate coordinate promoter activity;
(v) use of the invention to mark cells or cell structures;
(vi) use of the invention to characterize gene expression;
(vii) use of the invention for drug discovery and
(viii) methods of generating new fluorescent proteins.

### Split Fluorescent Proteins (“SFPs”)

The term “split fluorescent protein” or “SFP,” as used herein, refers to a portion of a fluorescent protein (“FP”) which, when covalently or non-covalently combined with one or more complementary SFP, is fluorescent. The reconstituted form of the fluorescent protein, which may differ from a native form of the FP, is referred to herein as “reconstituted fluorescent protein” or “RecFP.” When SFPs from a given parent FP, such as GFP from A. Victoria, form a RecFP, the terminology may be adjusted to refer to the parent (e.g., “RecGFP”).

The number of complementary SFPs used to produce a RecFP is preferably two but may be more than two, e.g. 3, 4, etc. An SFP is preferably non-fluorescent, but it may be fluorescent provided that its emitted fluorescence, if any, is either less intense or at a different wavelength than that of RecFP. The intensity or wavelength of fluorescence emitted by a RecFP may be the same or different from that of any FP from which it is derived.

SFPs may be derived from any FP that is detectable in vivo without the presence of a separate enzymatic substrate or cofactor, particularly FPs having a “β-barrel” or “β-can” conformation structurally homologous to the GFP of A. victoria. Examples of FPs that may be used as the basis of SFPs, according to the invention, include but are not limited to GFP of A. Victoria and fluorescent variants thereof (e.g., S65T, EGFP), FPs known in the art as “cyan FPs” (“CFPs”), “yellow FPs” (“YFPs”, including “YFP Venus” (Nagai et al., 2002, Nature Biotechnol. 20:87-90)), “blue FPs” (“BFPs”), and “red FPs” (“RFPs”) (quotations employed because color designation may be subjective or condition dependent), circularly permuted FPs (Baird et al., 1999, Proc. Natl. Acad. Sci. U.S.A. 96:11241-11246), monomeric RFPs (e.g., see Campbell et al., 2002, Proc. Natl. Acad. Sci. U.S.A. 99:7877-7882 and Bevis and Glick, 2002, Nature Biotechnol. 20:83-87); pH sensitive FPs (e.g., pH sensitive GFP (“pHluorin”); Meisenbock et al., 1998, Nature 394:192-195), photoactivatable FPs (e.g., photoactivatable GFP (Patterson et al., 2002, Science 297:1873-1877), voltage sensitive FPs (e.g., “FlaSh” (Guerrero et al., 2002, Biophys. J. 83:3607-3618) and “SPARC” (Ataka et al., 2002, Biophys. J. 82:509-516) and FPs from marine coelenterates, including but not limited to Renilla mulleri, Heteractis crispa, Entacmaea quadricolor, Discosoma and Trachyphyllia geoffroyi (for additional references, see Zhang et al., 2002, Nat. Rev. Mol. Bio. 3:906-918, Sawano et al., 2000, Nucl. Acids Res. 28:E78; Griesbeck et al., 2001, J. Biol. Chem. 276:29188-29194; Nagai et al., 2002, Nature Biotechnol. 20:87-90; Scholz et al., 2000, Eur. J. Biochem. 267:1565-1570; Baird et al., 1999, Proc. Natl. Acad. Sci. U.S.A.; Deitrich and Maiss, 2002, Biotechniques 32: 286, 288-90, 292-3; Su et al., 2001, Biochem. Biophys. Res. Commun. 287(2):359-65 and other references cited herein).

In specific non-limiting embodiments, the present invention relates to SFPs which have, as parent, GFP from A. Victoria having an amino acid sequence as set forth at GenBank Acc. No. P42212. In other specific non-limiting embodiments, the present invention relates to SFPs which have, as parent, GFP that has an amino acid sequence that varies from the sequence set forth at GenBank Acc. No. P42212 at the following residues :F64L, S65C, Q80R, Y151P and 1167T (see Example Section 6, below). In further specific non-limiting embodiments, the present invention provides for RecFPs which comprise amino acid sequences that vary from GenBank Acc. No. P42212 as follows: F64L, S65C, Q80R, Y151L and 1167T; S65C and Q80R; Y66W, N1461, M153T and V163A; S65G, V68L, S72A and T203Y; S65G, V68A, S72A and T203Y. Still other non-limiting examples of FPs that may serve as parents of SFPs according to the invention are FPs having amino acid sequences set forth in the following GenBank Accession Numbers: 1G7KA, 1G7KB, 1G7KC, and 1G7KD (for four chains of RFP of Discosoma); AAC53684 (a GFP); AA048591 (a YFP); YP 008577 (a BFP); and CAD53293 (a CFP). The present invention further provides, in additional non-limiting embodiments, for SFPs based on FP parents that are at least about 90 percent and preferably about 95 percent homologous to the foregoing proteins, as determined using standard software for homology determination based on amino acid sequence.

The numbering of amino acid residues in FPs having β-barrel or β-can structures presented herein is based on an alignment between the FP sequence and GFP of Aequorea Victoria having GenBank Accession No. P42212 (SEQ ID NO: 1) based on sequence homology, as may be determined by standard techniques and software known in the art.

The FP may be split to produce two or more SFPs which may be reassociated to form a RecFP. Relative to the amino acid sequence of the FP upon which it is based, an SFP may be an N-terminal, C-terminal, or middle (“M”)—SFP, also referred to herein as NSFP, CSFP or MSFP, respectively. The term “complementary” refers to SFPs that may assemble or be made to assemble to form a RFP. Complementary SFPs may together account for the entire amino acid sequence of the FP on which they are based, or may constitute more or less amino acid sequence. For example, an NSFP may account for residues 1-155 of GFP and a complementary CSFP may contain residues 156-238 of that protein. Alternatively, an NSFP may comprise residues 1-173 of a FP, and a complementary CSFP may comprise residues 155-238, where the two can be assembled to form a RecFP (see Hu and Kerppola, 2003, Nature Biotechnol. 21:539-545); in this circumstance, there is a redundancy in FP amino acid sequence in the RecFP. Accordingly, the SFPs are functionally complementary.

Relative to the amino acid sequence of the parent FP, the SFP has at least one terminus (and possibly both) arising within the internal parent sequence, which is referred to herein as the “split point.” For example, the split point of GFP used to design a NSFP having amino acids 1-156 of GFP is 156. Not all complementary SFPs share the same split point. In the last example provided in the preceding paragraph, the NSFP has a split point of 173 whereas its complementary CSFP has a split point of 155.

For FPs that comprise a “β-barrel” or “β-can” structure it is desireable to split the protein so as to facilitate assembly of RecFP into an equivalent structure. In one set of non-limiting embodiments, the split point may occur in loops of the FP β-barrel structure. In a related embodiment, where the FP is a β-can comprising P sheet segments, a split point interrupts a β-sheet segment (rather than occurring at a junction between sheets). In preferred non-limiting embodiments of the invention, the split occurs between residues 140 and 180 (numbering according to GFP), preferably between residues 140-150, or between residues 1-55 and 175, or between residues 150-160, or between residues 155-160, or between residues 170 and 175, more preferably at residue 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174 or 175. The “split” may be accomplished, for example, by engineering a cDNA encoding FP to delete the regions of the FP to be omitted in the SFP. Of note, other regions of the FP may be altered by insertion, deletion, or substitution. Preferably, but not by way of limitation, the SFP is at least about 90 percent, more preferably, 95 percent, identical to the corresponding FP sequence considering all changes, as determined using standard homology software. For example, a NSFP based on a split point of 155 in the parent FP has an amino acid sequence that is at least about 90 percent and preferably at least about 95 percent identical to residues 1-155 of the parent FP. The differences can arise from insertion, deletion, or substitution of amino acids; for example, the sequence may be truncated at its N-terminus, so that the SFP has both termini different from its parent FP.

SFPs may be assembled to form a RecFP by a covalent or non-covalent linkage. Of a plurality of SFPs that assemble to form a RecFP, each SFP may be joined to a binder element (“SFP-binder”), where the plurality of binder elements can covalently or non-covalently join. Binder elements of complementary SFPs may be the same or different. For example, binder elements may be components of a homomeric or heteromeric protein. As another non-limiting example, binder elements may be components of a ligand/receptor pair. Examples of compatible binder elements include, but are not limited to, an antiparallel leucine zipper (as described in U.S. Patent Application Publication No. 2003/0003506); calmodulin/M13 (as described in Ozawa et al. 2001, Anal. Chem. 73:5866-5874); immunoglobulin (including single chain antibodies and portions thereof)/peptide ligand; hormone/receptor; clathrin, enzyme/substrate; integrins such as alphaIIb and beta3; ubiquitin/ubiquitin interacting motif; viral capsid proteins (e.g., see Barklis et al., 1998, J. Biol. Chem. 273:7177-7120) and other interacting proteins known in the art (e.g., see Xenarius, 2002, Nucl. Acids Res. 30:303-305 regarding the protein interaction database, “DIP” at http://dip.doe-mbi.ucla.edu; Han et al., Bioinformatics, PMID# 15117749 regarding the human protein interaction database http://www.hpid.org; and information available from Biomolecular Interaction Network Database (BIND), Cellzome (Heidelberg, Germany), Dana Farber Cancer Institute (Boston, Mass., USA), the Human Protein Reference Database (HPRD), Hybrigenics (Paris, France), the European Bioinformatics Institute's (EMBL-EBI, Hinxton, UK) IntAct, the Molecular Interactions (MINT, Rome, Italy) database, the Protein-Protein Interaction Database (PPID, Edinburgh, UK) and the Search Tool for the Retrieval of Interacting Genes/Proteins (STRING, EMBL, Heidelberg, Germany)). The binder element may be attached to an SFP at either terminus (and still is referred to herein as “SFP-binder”). The binder may, in the process of association, change structure; for example, the binder may comprise an intein together with a member of an interacting pair of proteins (as in Ozawa et al. 2001, Anal. Chem. 73:5866-5874); when the protein pair interact, splicing occurs via the inteins and the interacting pair are cleaved from the now covalently-joined RecFP. The binder element in such embodiments therefore comprises a member of an interacting set of proteins together with an adherent structure that forms a linkage when brought into proximity of a partner structure; in addition to an intein (which produces a covalent linkage), another non-limiting example of an adherent structure (that produces a non-covalent linkage) is a leucine zipper domain.

In addition, an SFP or SFP-binder molecule may be linked to a localization molecule (“LM”) that may direct the SFP to a particular cellular (or extracellular) compartment. Examples of LMs include nuclear localization signal, KDEL, signal peptides, synaptic vesicle proteins such as synaptobrevin, mitochondrial localization signals, peroxisomal localization signals, and the like. LMs may also be proteins characteristically found in particular cellular locations. Example 6 below presents results when complementary SFPs are directed to the nucleus. One, a plurality, or all complementary SFPs may be joined to an LM, depending on experimental design. The LM may be attached to either terminus of the SFP or SFP-binder molecule (to form SFP-LM or SFP-binder-LM).

Accordingly, the molecules that may assemble or be assembled to form RecFPs include SFP, SFP-binder, SFP-LM and SFP-binder-LM, which are collectively referred to herein as SFP-constructs. SFP-constructs that can assemble or be assembled to form a fluorescent RecFP are “complementary.” An SFP-construct may further comprise a linker molecule to provide a desirable distance or functional alignment between SFPs; such a linker molecule may be between 1 and 50 amino acids, and preferably between 10 and 20 amino acids, in length.

Standard laboratory methods may be used to confirm that SFP—constructs co-expressed in vivo form fluorescent RecFP.

### Nucleic Acids Encoding SFP-Constructs

The present invention provides for nucleic acid molecules encoding SFP-constructs.

For example, the present invention provides for a nucleic acid encoding a SFP (as defined supra, which may be a NSFP, MSFP or CSFP) that may further encode a binder element and or a localization molecule (“LM”). Such molecules may comprise, in preferred non-limiting embodiments, a promoter element operatively linked to nucleic acid encoding the SFP, binder element, and/or LM. Such nucleic acids may contain additional molecules associated with expression, such as a transcription termination signal, Shine Delgarno sequence, and so forth.

In alternative embodiments, the present invention provides for nucleic acid molecules than comprise nucleic acid encoding a SFP and/or a binder element and/or a LM, without a promoter sequence. Transcription of the comprised SFP construct may be directed by either the insertion of said nucleic acid downstream of an endogenous promoter in a host cell, or by the introduction of a exogenous promoter element, for example by genetic engineering techniques.

In further embodiments, a nucleic acid may comprise nucleic acids encoding two or more complementary SFPs, each optionally linked to a binder element and/or LM, said coding sequences optionally linked to a single promoter or to separate promoters (for each SFP-construct to be expressed).

Any of the foregoing nucleic acids may be comprised in an appropriate vector molecule. Suitable vectors include, but are not limited to, plasmid, phage, or viral vectors such as adenovirus, adeno-associated virus, vaccinia virus, retrovirus, or baculovirus.

### Host Cells and Organisms Containing SFP-Constructs

The present invention further provides for cells and organisms containing SFP-constructs.

In a particular set of non-limiting embodiments, the present invention provides for a cell containing a nucleic acid encoding a SFP-construct, as described in the preceding section. Said nucleic acid may be operably linked to an endogenous cell promoter or an exogenous promoter. Said nucleic acid may be expressed or may be transcriptionally silent. The cell may further contain a nucleic acid encoding one or more complementary SFP-constructs. The nucleic acid may be introduced into the cell by standard techniques, including transfection, electroporation, microinjection, via a vector, by the preparation of a transgenic organism, or by breeding organisms.

The cell may be a eukaryotic or a prokaryotic cell. It may be a cell of a unicellular, colonial or multicellular organism such as a bacteria, plant, protozoan, yeast, mold, fungus, or vertebrate or invertebrate animal. The cell may be a mature cell, an embryonic cell, a stem cell, an undifferentiated cell or a dedifferentiated cell. In specific non-limiting embodiments, the cell may directly or indirectly originate (e.g. in culture) in a nematode (e.g C. elegans), insect (e.g., Drosophila melanogaster), fish (e.g., Danio rerio (zebrafish)), amphibian (e.g. frog, toad or salamander), bird (e.g. chicken or quail), or a mammal, for example but not by way of limitation a rodent (e.g., mouse, rat, rabbit or woodchuck), an ungulate (e.g. sheep, goat, horse or cow), a pig, or a primate (e.g ape, monkey, or human).

The cell may be a member of a cell population, such as a cell culture, a tissue, an organ, or an organism. The cell population may further contain additional cells which do, or do not, contain a SFP-construct. In preferred non-limiting embodiments, the present invention provides for cell populations in which at least about 50, 60, 70,80, or 90 percent of the cell members contain an SFP-construct.

In certain non-limiting embodiments, the nucleic acid encoding the SFP construct is linked to an endogenous host or exogenous promoter which may be (i) active in the cell; (ii) an active or inactive tissue specific promoter; or (iii) inactive but capable of activation by an activating agent, including the gene product of a second promoter element. An “endogenous” promoter is a native promoter that is present in its normal genomic position in the cell, wherein nucleic acid encoding the SFP-construct was inserted downstream of the native promoter. An “exogenous” promoter is a promoter that was introduced together with the nucleic acid encoding the SFP-construct; it may be a promoter that is found in the cell in nature, a variant of such a promoter, or a promoter that is found in another type of organism (such as an organism of another species).

In specific non-limiting embodiments, the present invention provides for a cell population comprising cells that contain nucleic acid encoding a SFP-construct, without a complementary SFP-construct. In particular non-limiting embodiments, the cell population is an organism, preferably a multicellular organism. The organism may be mature or immature. An immature organism may be embryonic, fetal, neonatal, larval, or otherwise may not yet have achieved sexual maturity. Non-limiting examples of such cell populations include C. elegans, Drosophila melanogaster, Danio rerio (zebrafish), Mus musculus and other experimental mammals, chickens, quails and other experimental birds, Xenopus laevis, salamander and other experimental amphibians, slime mold cultures such as Dictyostelium discoideum, fungi, colonial algae, and plants. The organism may be a transgenic organism or the progeny thereof. Such cell populations and in particular organisms may be used as test systems into which one or more complementary SFP-construct may be introduced.

In alternative non-limiting embodiments, the present invention provides for cell populations, and in particular organisms, as set forth above, that comprise cells that contain nucleic acids encoding complementary SFP-constructs, wherein the expression of at least one SFP-construct is under the control of an inactive promoter and at least one SFP-construct is under the control of a promoter that is constitutively active in at least a subset of cells in the population. Such cell populations and organisms may be used to identify test agents that activate the inactive promoter.

In still other alternative embodiments, the present invention provides for cell populations, and in particular organisms, as set forth above, that comprise cells that contain nucleic acids encoding complementary SFP-constructs, in which at least one SFP-construct is under the control of a developmentally regulated promoter. Such organisms may be used in developmental biology studies.

### Use of the Invention to Demonstrate Coordinate Promoter Activity

The present invention may be used to demonstrate coordinate activity of promoters that control the expression of complementary SFP-conjugates. “Coordinate” as used herein means that the promoters are active within a period of time such that their SFP-conjugate products co-exist and are capable of assembling to form RecFP. The use of the term “coordinate” does not require that there be any dependence or direct or indirect functional relationship between the activity of the promoters, although in specific non-limiting examples of the invention, such dependence or relationship may exist. “Coordinate” need not mean “contemporaneous.” Moreover, because SFP-conjugates or RecFPs may be relatively unstable, promoters may be sequentially active, but if there is an interval between their activity that permits the degradation of SFP-conjugate and/or RecFP, their coordinate activity may not be detectable.

Thus, in a host cell containing complementary SFP-constructs under the control of different promoters, the promoters may be coordinately expressed if both promoters are active in the host cell type (e.g., tissue specific promoters, constitutively active promoters of “housekeeping” genes) or under conditions to which the host cell is exposed (e.g., changing developmental conditions, changes in extracellular environment, exposure to cytokines), including if one promoter is dependent on the gene product of the other for activity.

Thus, in particular, non-limiting embodiments, the present invention provides for a method of detecting coordinate activity of a first and a second promoter element in a host cell containing a first nucleic acid comprising the first promoter operably linked to a nucleic acid encoding a first SFP-construct and a second nucleic acid comprising the second promoter operably linked to a second nucleic acid encoding a second SFP-construct, where the first and second SFP-constructs are complementary, comprising detecting the formation of a RecFP from the SFP-constructs, for example by detecting fluorescence characteristic of the RecFP. The promoters may be different or the same, but preferably the promoters are different.

The present invention further provides for detecting coordinate activity of more than two promoters. For example, the method set forth above may be altered so that more than two complementary SFP-constructs are required to form RecFP. Alternatively, multiple pairs of promoter activity may be detected by practicing the method set forth in the preceding paragraph for each pair, wherein the RecFPs produced by each pair produce a distinctive fluorescence emission wavelength.

### Use of the Invention to Mark Cells or Cell Structures

The present invention provides for the marking of cells or cell structures by introducing RecFPs. The cells to be marked may be isolated or part of an organized cell population such as a tissue, organ, colony or organism. Cell structures that may be marked include intracellular structures such as the nucleus, nucleolus, mitochondria, endoplasmic reticulum, Golgi body, lysosome, storage vesicles, membrane and cytoskeleton. as well as extracellular structures such as released particles, the extracellular space, and the extracellular surface of the cell membrane. The present invention may be used to study the process of infection; for example, self-associating viral proteins may serve as binder elements between complementary SFPs such that viral assembly results in formation of RecFP, or a pathogen may contain, in its genome, a SFP-construct complementary to SFP-constructs encoded by a host cell.

As demonstrated in Example 6, below, the present invention enables the use of RecFPs, expressed from coordinately active promoters, to mark specific types of cells or cell structures. By depending on coordinate promoter activity for the generation of RecFPs, the invention provides an improvement over, for example, the expression of intact FP from a single promoter because frequently expression of a promoter is not restricted to a single cell type. Additionally, there is not always a promoter known that is specifically expressed only in one type or family of cell. The present invention allows the use of multiple promoters, which may be each expressed in a number of cell types, to mark only the specific type of cell or cell family in which all promoters are active.

Accordingly, the present invention may be used to mark cells in a population, which may have the following non-limiting utilities. In a cell culture, cells expressing complementary SFP-constructs and producing RecFPs may be identified by fluorescent microscopy and may be collected by fluorescence activated cell sorting. In a tissue, organ, or organism, a particular type of cell may be marked to study, for example, its development or changes in anatomical relationships with other cells. Also, in a specific non-limiting embodiment, different cells in a population may express individual SFP-constructs of a complementary pair, and the formation of RecFP may be an indicator of cell-cell fusion (for example, between HIV-infected cells, during conjugation of bacteria or in plasmodium phase of a slime mold).

Via LMs or binder elements, the SFP-constructs may be localized in a particular cellular structure. The localization of RecFP in the cell nucleus may be used to monitor nuclear morphology, passage into S-phase or nuclear fragmentation. The localization of RecFPs in lysosomes may be used to study changes in lysosome size. The localization of RecFPs in neural vesicles and the extracellular space may be used to study the dynamics of neurochemical release.

### Use of the Invention to Characterize Gene Expression

The present invention may be used to characterize the expression of a particular gene. As one specific non-limiting example, the cell type in which a particular gene is expressed may be determined by introducing, into a cell, a first nucleic acid encoding a SFP-construct operably linked to the promoter of the gene of interest, and a second nucleic acid encoding a complementary SFP-construct, operably linked to a promoter that is known to be active in that cell. Production of RecFP in the cell is indicative that the gene of interest is expressed in the cell.

Analogous methods may be used to determine the developmental period in which the gene of interest is expressed. A nucleic acid operably linked to the promoter of the gene of interest may be introduced into a cell together with a nucleic acid encoding a complementary SFP-construct operably linked to a promoter that is active during a particular developmental period. Production of RecFP during that developmental period indicates that the gene of interest is also expressed during the developmental period. It should be noted, however, that such a result may not be conclusive that the promoters are contemporaneously active, as, depending on the stability of the SFP-constructs, a given promoter may no longer be active but the corresponding SFP-construct may nevertheless persist in the cell.

In analogous methods, the present invention may be used to identify temporal relationships between promoters outside of the developmental period, for example in response to an environmental alteration, infection, exposure to a chemical agent or aging. For example, but not by way of limitation, a cell may comprise a first SFP-construct operably linked to an active promoter, and a second complementary SFP-construct operably linked to a regulated promoter; when the regulated promoter switches on RecFP may be produced, and when the promoter switches off, RecFP may diminish according to the half-life of the RecFP or its component SFPs. It may be an advantage, in such embodiments, that certain RecFPs have been observed to have a half-life shorter than parent FP (see Example Section 6, below), as such (relatively speaking) labile RecFPs permit better resolution for detecting a decrease in promoter activity.

The cell in the foregoing methods may be a cell in a cell culture, tissue, organ, or organism. It should be noted that in this description, where “introduction” of nucleic acid into a cell is recited, the skilled artisan would readily understand that an equivalent method could utilize a cell that already contained one or both SFP-construct nucleic acids, for example, a cell in a transgenic animal, and/or a cell in an animal that is the offspring of parents each carrying, in their genome, nucleic acid encoding one of the complementary SFP-constructs.

One specific non-limiting embodiment of the invention provides for the production of a set of tester strains in which NZGFP, NZYFP, and NZCFP are expressed from characterized promoters. These strains could be mated with animals expressing CZCFP from a promoter whose expression had not yet been characterized. With the color coding provided by the different NZ fluorescent proteins, relatively few (perhaps less than thirty) strains could be used to characterize gene expression in all of the 302 C. elegans neurons (118 classes). Similar “identikits” could be constructed for Drosophila, zebrafish, mice, and other organisms.

### Use of the Invention for Drug Discovery

The present invention provides for methods of identifying compounds that activate a promoter of a gene of interest. Such methods comprise exposing a cell containing nucleic acids encoding complementary SFP-constructs, where at least one of the promoters controlling expression of an SFP-construct is inactive, to a test agent, and then detecting whether or not RecFP is produced, where production of the RecFP indicates that the inactive promoter is directly or indirectly activated by the test agent. The cell may be an isolated cell or may be comprised in a cell culture, tissue, organ or organism.

The present invention offers the further advantage that cells in which RecFP is formed may be specifically identified, studied by fluorescence microscopy, and/or collected, for example by fluorescence activated cell sorting. In the latter case, the cells collected cells may be subjected to further analysis; for example, RNA may be collected from the cells that may be used to identify changes in the expression levels of various genes, and/or to produce an expression library.

Analogous methods may be used to identify agents that alter the development profile, tissue/cell type of expression, or intracellular or extracellular location of a gene, using variations of methods set forth in preceding sections.

Analogous methods may be used to identify compounds that affect coordinate promoter activity, in which the feature to be detected is the absence or decreased production of RecFP.

### Methods of Generation New FPs

The present invention further provides for methods of identifying new FPs having desirable properties by generating, from among complementary SFPs carrying various mutations relative to a parent FP, RecFPs comprising novel combinations of mutations and then identifying RecFPs having particularly useful properties. The mutations contained in the superior RecFPs may then be engineered into the parent FP molecule. Where conformational spacing between SFPs may be a significant component in the enhanced properties of the RecFP, one or more peptide spacer molecule (for example, but not by way of limitation, between 1 and 30 amino acids long) may be inserted into the parent FP molecule to produce a similar conformation.

In a non-limiting embodiment, the present invention provides for a FP comprising the following covalently linked amino acid sequence (SEQ ID NO: 1):

MSKGEELFTGVVPILVELDGDVNGHKFSVSGEGEGDATYGKLTLKFICTT
GKLPVPWPTLVTTFGYGLQCFARYPDHMKQHDFFKSAMPEGYVQERTIFF
KDDGNYKTRAEVKFEGDTLVNRIELKGIDFKEDGNILGHKLEYNYNSHNV
YIMADKQKNGIKANFKIRHNIEDGSVQLADHYQQNTPIGDGPVLLPDNHY
LSTQSALSKDPNEKRDHMVLLEFVTAAGITHGMDELYK.

A RecFP carrying these mutations was identified in Example 6 as having particularly advantageous fluorescent properties. The present invention further provides for a nucleic acid encoding the above amino acid sequence, and said nucleic acid operably linked to a suitable promoter element.

In addition, RecFPs having desirable properties identified by this method (for example, which have brighter fluorescence, or have unique excitation/emission characteristics) may be used as as reporter genes in contexts analogous to GFP itself. In specific, non-limiting embodiments, the SFP-constructs used to produce such superior RecFPs may be expressed off either the same promoter, each may be linked to a separate copy of the same type of promoter, or they may be expressed off different promoters.

### EXAMPLE

### Combinatorial Marking of C. Elegans Cells with Split Fluorescent Proteins

Expression of GFP and other fluorescent proteins depends on cis regulatory elements. Because these elements rarely direct expression to specific cell types, GFP production cannot always be sufficiently limited. The working example that follows demonstrates that reconstitution of GFP, YFP, and CFP previously split into two polypeptides yields fluorescent products when coexpressed in C. elegans. Because this reconstitution involves two components, it can confirm cellular coexpression and identify cells expressing a previously uncharacterized promoter. By choosing promoters whose expression patterns overlap for a single cell type, animals were produced with fluorescence only in those cells. Furthermore, when one partial GFP polypeptide was fused with a subcellularly localized protein or peptide, this restricted expression resulted in the fluorescent marking of the cellular components in a subset of cells.

### MATERIALS AND METHODS

### Nematode Maintenance

Animals were cultured at 20° C. as described (1) unless otherwise indicated. Wild type (N2) and the unc-4(e120) and unc-37(e262) mutants have been described (Brenner, 1974, Genetics 77: 71).

### Expression Constructs and Transformation

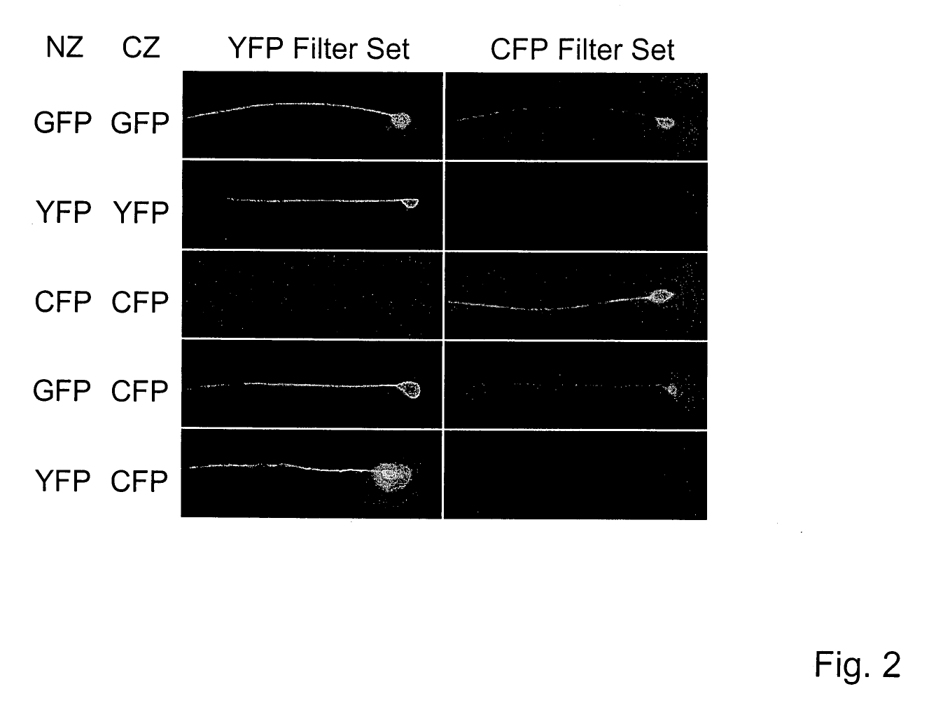

Bacterial expression plasmids for NZGFP and CZGFP (Ghosh et al., 2000, J. Am. Chem. Soc. 122: 5658) were gifts from Lynne Regan. The GFP sequence encoded by these plasmids differs from that of GFP listed as GenBank Acc. No. P42212 (SEQ ID NO: 1) in the following ways :F64L, S65C, Q80R, Y151P and 1167T (which Ghosh et al., 2000 had reported, except that they reported the 167 variation to be I167P). The coding sequences of NZGFP and CZGFP were amplified by PCR with primers that introduced 5′ BamHI and 3′ EcoRI sites (these and the other primers used in this study are given in Table 1; the resulting plasmids are given in Table 2). The resulting PCR products were cut with BamHI and EcoRI, and cloned into Fire promoter-less GFP plasmid pPD95.77 (all the Fire vectors used in these studies are described at www.ciwemb.edu/pages/firelab.html). This procedure essentially replaced the original coding region of GFP in pPD95.77 with nzgfp or czgfp. pPD95.77 has artificial introns in the 5′ UTR, the GFP coding sequence, and the 3′ UTR that appear to stimulate GFP expression. It was found that one intron in the GFP coding sequence (nucleotides 724-774) differed in several places from the sequence reported on the above website for pPD95.77. The reported sequence was gtaagtttaaacttggacttactaactaacggattatatttaaattttcag (SEQ ID NO:2) and the sequence used herein was found to be gtaagtttaaacAtgATTttactaactaacTAatCTGatttaaattttcag (SEQ ID NO:3). All these constructs contain the 3′ UTR intron; addition of other introns to nzgfp and czgfp did not significantly improve fluorescence. For FIG. 2, constructs using all the Fire introns were used. The GFP sequence used for these constructs (from pPD95.77) has the S65C and Q80R mutations, but none of the other changes found in the Ghosh et al. constructs.

TABLE 1
Primers for PCR Amplification

| | |
|---|---|
| Sequence | Oligonucleotides |

| | |
|---|---|
| nzgfp^{a} | 5′ primer CGCGGATCCATGGCTAGCAAAGGAGAAGAACT |
| | (SEQ ID NO:4) |
| | 3′ primer CCGGAATTCTCACTGAGCCAGTTCTTTCTTC |
| | A |
| | (SEQ ID NO:5) |
| czgfp^{a} | 5′ primer CGCGGATCCATGGCTAGCGCACAGCTGG |
| | (SEQ ID NO:6) |
| | 3′ primer CCGGAATTCTCAGTTGTACAGTTCATCCATGC |
| | C |
| | (SEQ ID NO:7) |
| ngfp^{a} | 5′ primer CGCGGATCCATGGCTAGCAAAGGAGAAGAACT |
| | (SEQ ID NO:8) |
| | 3′ primer CCGGAATTCTCAGCCAGAGCCAGAGCCACCTT |
| | (SEQ ID NO:9) |
| P_{unc-4} | 5′ primer GATCAAGCTTCCCCAAATTGGAACAGTGAAAT |
| | AC |
| | (SEQ ID NO:10) |
| | 3′ primer GATCGGATCCCATTTTCACTTTTTGGAAGAAG |
| | AAG |
| | (SEQ ID NO:11) |
| P_{acr-5} | 5′ primer CATGTGATTATGCATGCGAAAG |
| | (SEQ ID NO:12) |
| | 3′ primer GCATGCTGAAAATTGTTTTTAAAGC |
| | (SEQ ID NO:13) |
| P_{unc-47} | 5′ primer GTACAAGCTTGACAAAACAACTTTCTTGG |
| | (SEQ ID NO:14) |
| | 3′ primer GTACGGATCCATTTGATCCTGGAACATAGAT |
| | AATTTG |
| | (SEQ ID NO:15) |
| P_{mec-18} | 5′ primer TGAAATAAGCTTCAATTAATTCGTCTA |
| | (SEQ ID NO:16) |
| | 3′ primer CGCGGATCCCATGCTCACAACCTTCTTGGAAG |
| | G |
| | (SEQ ID NO:17) |
| P_{mec-2} | 5′ primer AAGCTTGCATGCCTGCAGTAACATTT |
| | (SEQ ID NO:18) |
| | 3′ primer CGCGGATCCCATAGATTGAATGTGTGGTGCAT |
| | TCAG |
| | (SEQ ID NO:19) |
| P_{unc-24} | 5′ primer CGCAAGCTTGAAGCTCTCGGAAA |
| | (SEQ ID NO:20) |
| | 3′ primer CGCGGATCCCATTACACTTTGACTTGGATCAC |
| | C |
| | (SEQ ID NO:21) |
| P_{egl-44} | 5′ p1rimer CGCGGATCCATAGGAGTTCCCTCTGACTTC |
| | GC |
| | (SEQ ID NO:22) |
| | 3′ primer CGCGGATCCCATAATCTTGAAATAAGAACTGG |
| | GTA |
| | (SEQ ID NO:23) |
| Pₛₜₒ₋₆ | 5′ primer ACGCGTCGACTGGACCACCAGCTTGCAGT |
| | (SEQ ID NO:24) |
| | 3′ primer CGCGGATCCCATGTTTTGTCGGCTCCTAAAAC |
| | (SEQ ID NO:25) |
| snb-1 | 5′ primer CGCGGATCCGACGCTCAAGGAGATGCCGGC |
| | (SEQ ID NO:26) |
| | 3′ primer CGCGGATCCTTTTCCTCCAGCCCATAAAAC |
| | (SEQ ID NO:27) |
| nzgfp | 5′ primer: CTATAACTCACACAATGTATACATCATGGCA |
| and | GACAAACAAGGTGGCTCTGGCTCTGGCGC |
| nzyfp^{b} | (SEQ ID NO:28) |
| | 3′ primer: ACCGGCGCTCAGTTGGAATTCTACGAATGCT |
| | ACTGAGCCAGTTCTTTCTTCAGTGCC |
| | (SEQ ID NO:29) |
| czgfp | 5′ primer: ATTTTCAGGAGGACCCTTGAGGGTACCGGTA |
| and | GAAAAAATGGCTAGCGCACAGCTGG |
| czyfp^{b} | (SEQ ID NO:30) |
| | 3′ primer: GTAAAATCATGTTTAAACTTACAACTTTGAT |
| | TCCATTCTTACCGCTTCCACCCTGTGCC |
| | (SEQ ID NO:31) |
| nzcfp^{b} | 5′ primer: CTATATTTCACACAACGTATACATCACTGCC |
| | GACAAACAAGGTGGCTCTGGCTCTGGCGC |
| | (SEQ ID NO:32) |
| | 3′ primer: ACCGGCGCTCAGTTGGAATTCTACGAATGCT |
| | ACTGAGCCAGTTCTTTCTTCAGTGCC |
| | ((SEQ ID NO:33) |
| czcfp^{b} | 5′ primer: ATTTTCAGGAGGACCCTTGAGGGTACCGGTA |
| | GAAAAAATGGCTAGCGCACAGCTGG |
| | (SEQ ID NO:34) |
| | 3′ primer: GTAAAATCATGTTTAAACTTACCGCTTTGAT |
| | CCCATTCTTACCGCTTCCACCCTGTGCC |
| | (SEQ ID NO:35) |
| 3Xnls | 5′ primer: GCGGGATCCACCGCCCCAAAGAAGAAACGCA |
| | AAGTACCGAGCTCAGAAAAAATGACC |
| | (SEQ ID NO:36) |
| | 3′ primer: GACTGGCTAGCCATTTTTTCTACCGGTACTT |
| | TGCGTTTCTTT |
| | (SEQ ID NO:37) |

^{a}Sequences were amplified from the bacterial clones of Ghosh et al., 2000, J. Am. Chem. Soc. 122: 5658.
^{b}Sequences were amplified from the bacterial clones of Ghosh et al., 2000, J. Am. Chem. Soc. 122: 5658 and used as megaprimers with the appropriate Fire vectors.

TABLE 2
Plasmid List^{a}

Plasmid Contents

| | |
|---|---|
| TU#707 | nzgfp |
| TU#708 | czgfp |
| TU#709 | ngfp |
| TU#710 | nzgfp^{b} |
| TU#711 | czgfp^{b} |
| TU#712 | nzyfp^{b} |
| TU#713 | nzyfp^{b} |
| TU#714 | nzcfp^{b} |
| TU#715 | czcfp^{b} |
| TU#716 | P_{mec-18}nzgfp |
| TU#717 | P_{mec-18}czgfp |
| TU#718 | P_{mec-18}nzyfp^{b} |
| TU#719 | P_{mec-18}czyfp^{b} |
| TU#720 | P_{mec-18}nzcfp^{b} |
| TU#721 | P_{mec-18}czcfp^{b} |
| TU#722 | P_{mec-2}czgfp |
| TU#723 | P_{mec-3}nzgfp |
| TU#724 | P_{unc-24}gfp |
| TU#725 | P_{unc-24}nzgfp |
| TU#726 | P_{hsp-16.2}nzgfp |
| TU#727 | P_{hsp-16.2}czgfp |
| TU#728 | P_{egl-44}czgfp |
| TU#729 | Pₛₜₒ₋₆gfp |
| TU#730 | Pₛₜₒ₋₆czgfp |
| TU#731 | Pₛₜₒ₋₆snb-1::czgfp |
| TU#732 | Pₛₜₒ₋₆3Xnls::czgfp |
| TU#733 | P_{unc-4}nzgfp |
| TU#734 | P_{unc-4}czgfp |
| TU#735 | P_{acr-5}nzgfp |
| TU#736 | P_{unc-47}nzgfp |

^{a}All the plasmids were based on Fire vector pPD95.77, which contains a GFP-coding sequence with several artificial introns. Unless indicated, the derived vectors replace this sequence with a coding sequence without introns.
^{b}The GFP-coding sequences in these plasmids were derived from Fire vector pPD95.77 and have artificial introns.

Split YFP and CFP plasmids were made by first replacing the GFP coding sequence in pPD95.77 with YFP coding sequence from pPD133.58 (although it was found that this plasmid contained a V68L change and not V68A listed on the website) or CFP coding sequence from pPD 133.51 using the fluorescent protein-coding AgeI—EagI fragment. Then, megaprimers (Brons-Poulsen et al., 1998, Mol. Cell Probes 12: 345) were made by amplifying the linker and zipper encoding regions of nzgfp and czgfp and used the Quikchange mutagenesis kit (Stratagene, La Jolla, Calif.) to add them to pPD95.77. The primers were constructed so that amplification of pPD95.77 simultaneously deleted the unwanted fluorescent protein coding sequence and maintained the presence of all the artificial introns. These constructs produce YFP containing the same mutations (S65G, V68L, S72A and T203Y) as 10C of Ormö et al.,1996, Science 273:1392-1395 (the Fire vector website, however, lists the V68L change as V68A) and CFP containing the mutations Y66W, N1461, M153T, V163A) used by Miller, 3rd et al., 1999, Biotechniques 26: 914. This CFP sequence is W7 (Heim and Tsien, 1996, Curr. Biol. 6:178-182), although it is lacking the N212K mutation. Protein-coding DNA sequences were verified (GeneWiz, Inc., North Brunswick, N.J.).

The following promoter sequences (upstream sequences to the start codon) were obtained from genomic DNA or appropriate Fire (pPD) vectors using PCR primers that introduced the indicated restriction sites: acr-5 (4.4 kb SphI-SphI fragment), egl-44 (3.1 kb BamHI-BamHI fragment), mec-2 (2.5 kb PstI-BamHI fragment), mec-3 (1.9 kb PstI-BamHI fragment from pPD57.56), mec-18 (0.4 kb HindIII-BamHI fragment), hsp16.2 (0.4 kb SphI-BamHI fragment from pPD49.78), sto-6 (2 kb SalI-BamHI fragment), unc-4 (2.5 kb HindIII-BamHI fragment), unc-24 (1.2 kb HindIII-BamHI fragment), unc-47 (1.7 kb HindIII-BamHI fragment). In cases of non-directional cloning, the correct orientation was verified by restriction digests. The entire genomic coding sequence of synaptic marker, snb-1, was amplified from pMN100.2 (a gift from Mike Nonet) and a BamHI site was added before its start codon and stop codon. This fragment was cloned into the Pₛₜₒ₋₆czgfp construct at the BamHI site such that snb-1 was downstream of the sto-6 promoter and in frame with czgfp. The orientation and sequence of snb-1 coding region were verified. The sequence containing three tandem repeats of the SV40 nuclear localization signal (3Xnls) was amplified from Fire vector pPD136.15 using primers that introduced 5′ BamHI and 3′ NheI sites. The amplified BamHI-NheI fragment was cloned into Pₛₜₒ₋₆czgfp such that the 3Xnls sequence was in frame with the downstream czgfp sequence. The sequence of this localization signal was verified.

Transgenic animals were generated by microinjection using the pRF4 dominant roller plasmid (50 μg/ml) as a transformation marker (Mello et al., 1991, EMBO J. 10: 3959). Expression plasmids were used at 50 μg/ml if injected alone or 25 μg/ml if two were injected. At least three stable lines were obtained for each genotype. All lines produced animals with similar fluorescence. When split GFP expression from the egl-44 and mec-3 promoters was measured, 5 μg/ml of the P_{mec-3}nzgfp and 45 μg/ml of the P_{egl-44}czgfp were used because higher concentrations of P_{mec-3}nzgfp resulted in occasional fluorescence in touch receptor neurons.

### Stability of RecGFP

An integrated line carrying P_{unc-4}gfp was generated with γ ray irradiation. An integrated line carrying P_{unc-4}nzgfp and P_{unc-4}czgfp was generated by a spontaneous integration event. Both lines were maintained at 25° C. Animals were synchronized by collecting newly hatched larvae (within 2 hr) from plates from which larvae and adults had been removed with distilled water. The number of fluorescent ventral cord cell bodies was determined using epifluorescence at <2 hr (hatching), ˜20 hr (L2/L3 larvae), and ˜40 hr (L4 larvae/young adults).

### Microscopy

Living L4 and young adult nematodes were viewed after being mounted on agarose pads (2% agarose, 50 mM Tris HCl, pH 8.5, 5 mM MgCl2). For heat shocking L4 or young adults were incubated at 32° C. for two hours, transferred to 20° C., and viewed after approximately 12 hr. Animals were viewed by epifluorescence using a Zeiss Axioskop 2 microscope equipped with the following filter sets (Chroma Technology Corp., Rockingham, Vt.): (1) GFP: excitation D480/30x, dichroic 505DCLP, emission D605/55m; (2) YFP: excitation HQ500/20x, dichroic Q515LP, emission HQ520LP; (3) CFP: excitation D436/20x, dichroic 455DCLP, emission D480/40m. Photographs were taken by a SPOT digital camera (Diagnostic Instruments, Inc., Sterling Heights, Mich.).

### RESULTS

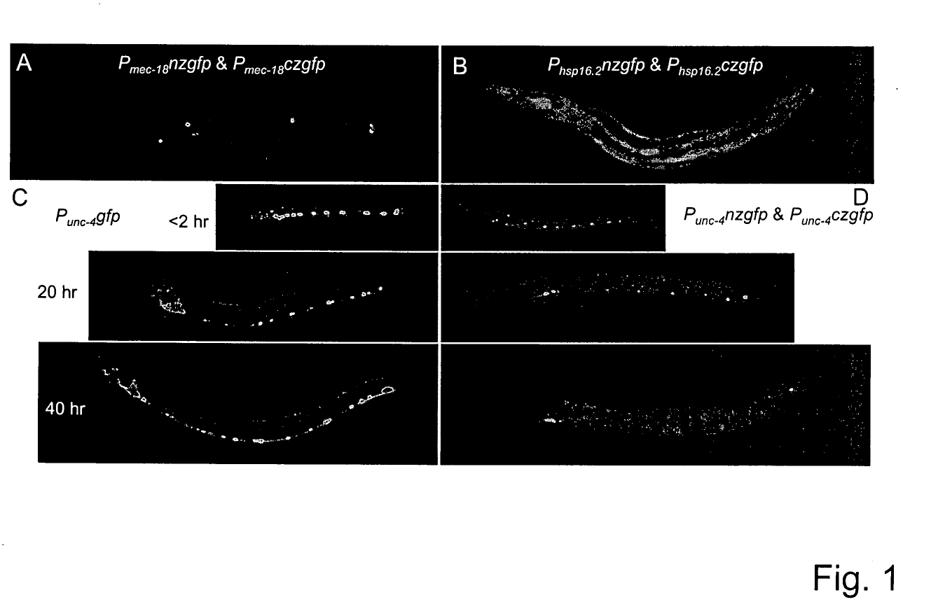

NZGFP and CZGFP polypeptides were expressed from the promoter for the mec-18 gene (P_{mec-18}) of C. elegans. This promoter is only expressed in the six touch receptor neurons of this animal. Bright fluorescence was visible in these neurons when animals expressed both split GFP/leucine zipper polypeptides from this promoter (P_{mec- 18nzgfp and P}_{mec-18}czgfp; FIG. 1A), but not when either NZGFP or CZGFP was expressed alone. This fluorescence did not result from DNA rearrangement during C. elegans transformation because no fluorescence was seen in animals expressing P_{mec- 18}nzgfp and czgfp, i.e., when CZGFP is not expressed from P_{mec-18}. Furthermore, the absence of CZ prevented the production of fluorescence.

RecGFP fluorescence was not promoter or tissue dependent, since it could be generated using the hsp 16.2 heat shock promoter (FIG. 1B), which is widely expressed, and the unc-4 promoter, which is reported to be expressed in four types of motor neurons (SAB, VA, DA, and VC) (Lickteig et al., 2001, J. Neurosci. 21 2001; Miller, 3^{rd} and Niemeyer, 1995, Development 121: 2877) (FIGS. 1C and D).

The expression from the unc-4 promoter revealed an unusual and potential useful characteristic of the RecGFP: it appeared to have a relatively shorter half-life compared to GFP. The unc-4 gene is transiently expressed in different motor neurons at various times in C. elegans development. Because of the stability of GFP, this transient expression cannot be appreciated when complete GFP is used as a marker; young adult animals (2-3 d post hatching) contain fluorescent cells that have expressed GFP in the embryo, early larva, and late larva (Poyurovsky et al., 2003, Mol. Cell 12: 875). In contrast, the only cells that fluoresce in young adults expressing a rapidly degraded GFP 5 (caused by the fusion of the RING finger domain from the E3 ubiquitin ligase Mdm2) are the late larval cells (Poyurovsky et al., 2003, Mol. Cell 12: 875). The animals with RecGFP also displayed a similar loss in fluorescence as they matured (FIGS. 1C-D).

The ability to form a reconstituted fluorescent protein was not restricted to split GFP, but was also observed in experiments using split YFP and split CFP (FIG. 2). In addition, it was found that CZCFP (i.e., CZGFP with the CFP mutation V163A) can be used generally with various forms of NZ fluorescent protein fusions. Fluorescence from RecGFP was seen with both the Chroma YFP and CFP filter sets, whereas RecYFP and RecCFP were detected only with the appropriate filter set. The reconstituted fluorescent protein from NZGFP and CZCFP (RecG/CFP was detected with both filter sets (although stronger with the YFP filter set). In contrast, the reconstituted fluorescent protein from NZYFP and CZCFP (RecY/CFP) was easily detected with the YFP filter set, but barely detectable with the CFP filter set. This last combination gave the most intense fluorescence of any of the combinations tested (FIG. 2). Combinations of NZCFP and NZGFP with CZYFP resulted in little or no fluorescence.

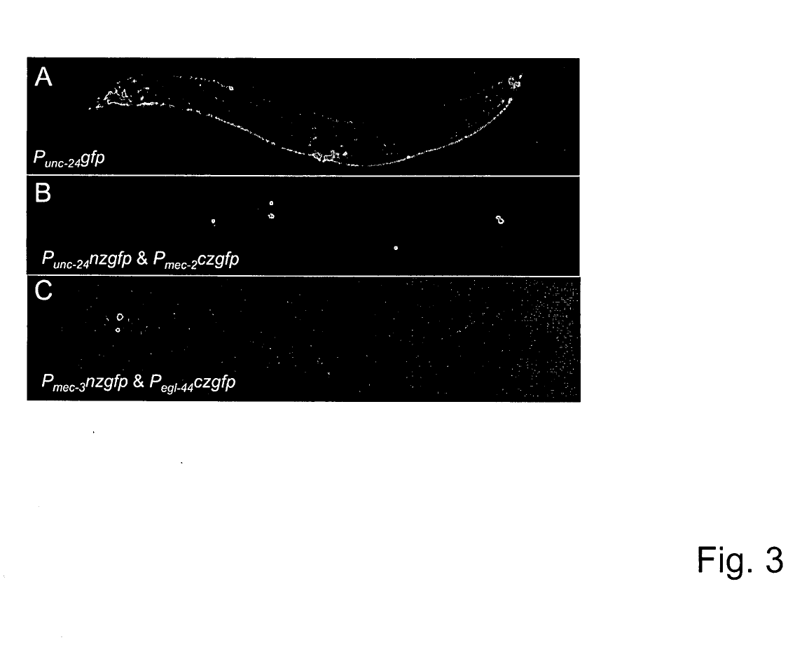

To demonstrate that RecGFP can identify cells that coexpress different promoters, NZGFP was expressed from the unc-24 promoter and CZGFP from the mec-2 promoter. The unc-24 promoter is expressed in the C. elegans touch receptor neurons and in many cells in the ventral cord (FIG. 3A); the mec-2 promoter is expressed in the six touch receptor neurons. RecGFP, with components expressed from the unc-24 and—mec-2 promoters, was found only in the six touch receptor neurons (FIG. 3B), demonstrating the increased specificity obtained using the present invention.

Because RecGFP formation requires the combinatorial expression of two promoters (it acts as an “and” gate), it can overcome the limitation that GFP expression is dependent on available regulatory elements. To demonstrate the additional restriction 30 possible with RecGFP, animals were generated in which only the two FLP neurons fluoresced. No FLP-specific promoter has been reported, but mec-3 and egl-44, genes that are expressed in several different cell types, are coexpressed only in these neurons (Way and Chalfie, 1989, Genes Dev. 3: 1823; Wu et al., 2001, Genes Dev. 15: 789). By expressing NZGFP from the mec-3 promoter and CZGFP from the egl-44 promoter, animals with labeled FLP neurons were obtained (FIG. 3C).

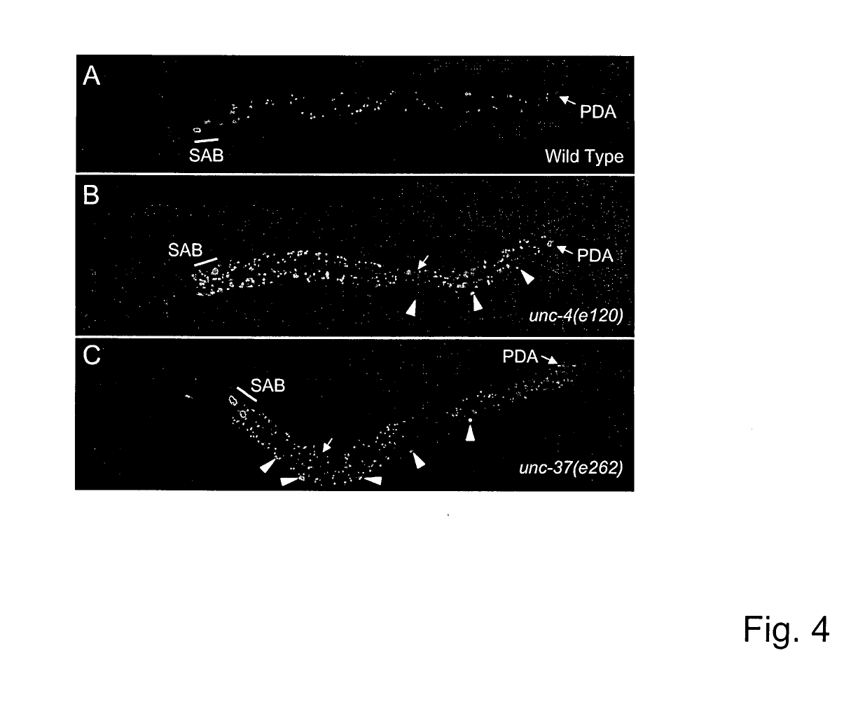

The ability of RecGFP to visualize coexpression can also be used to demonstrate changes in gene expression. To demonstrate this utility, the effects of mutations in the genes for the homeodomain transcription factor UNC-4 and the groucho-like transcription factor UNC-37 on the fate of motor neurons were examined. Previously, Winnier et al. (Winnier et al., 1999, Genes Dev. 13: 2774) showed that mutations in unc-4 and unc-37, which are expressed in and determine the fate of VA motor neurons, caused additional cells in the ventral cord to express the acr-5 gene. As shown in FIG. 4, this finding has been confirmed and demonstrated directly. RecGFP from P_{unc-4}nzgfp and P_{acr-5}czgfp formed in several ventral cord neurons in unc-4 and unc-37 mutants, but not in wild type (FIG. 4); these cells are the VA motor neurons. It was also found that several unc-4-expressing cells outside of the ventral cord (specifically, the SAB neurons and a cell we have tentatively identified as PDA) expressed acr-5 even in wild-type animals. Interestingly, the intensity of fluorescence in these cells was brighter in the mutants than in wild-type animals. Because acr-5 is expressed in many cells, these observations could not have been easily made using coexpression of different color fluorescent proteins. In addition to assessing effects of known mutations, animals expressing these and similar constructs could be used to identify new mutations, growth conditions, or reagents that change cell fate or gene expression.

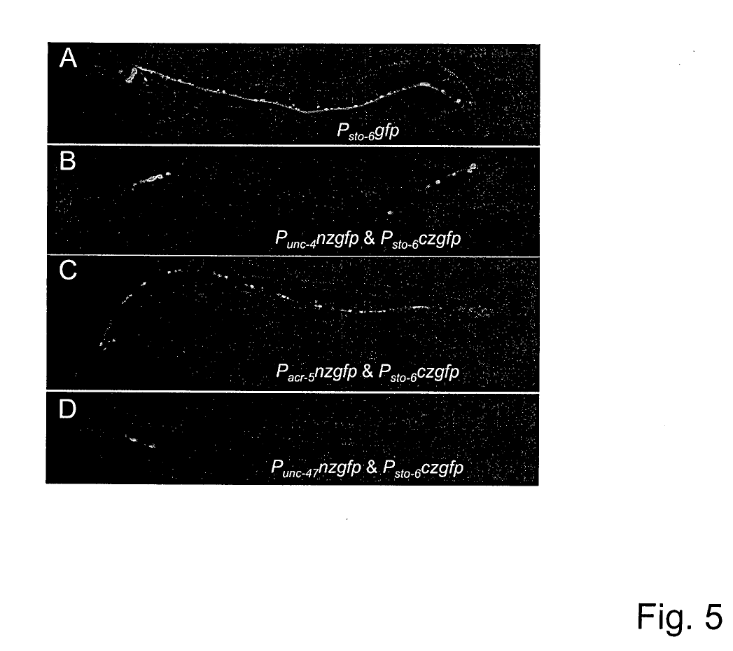

The combinatorial action of split GFP can also be used to identify cells expressing a particular gene. To demonstrate this property, we examined the expression of the C. elegans sto-6 gene, a stomatin-encoding gene whose expression had been previously uncharacterized. Pₛₜₒ₋₆gfp is expressed in many of the motor neurons of the ventral cord (FIG. 5A). To discover which neurons expressed sto-6, we used promoters that were known to be expressed in different classes of motor neurons in the ventral cord. We obtained split GFP fluorescence from Pₛₜₒ₋₆czgfp when NZGFP was generated from the unc-4 and acr-5 promoters, but not when it was generated from the unc-47 promoter (FIGS. 5B-D). These results indicate that sto-6 is expressed in the ventral cord in the excitatory motor neurons [the VA, DA and possibly VC neurons that express unc-4 (Lickteig et al., 2001, J. Neurosci. 21: 2001; Miller, ₃^{rd} and Niemeyer, 1995, Development 121: 2877) and the VB and DB motor neurons that express acr-5 (Winnier et al., 1999, Genes Dev. 13: 2774)], but not the inhibitory motor neurons [the VD and DD motor neurons that express unc-47 (McIntire et al., 1997, Nature 389: 870)].

The apparent short half-life of RecGFP raises an important caution about negative results in these experiments: promoters that are expressed at different times in the same cells may not produce a fluorescent product if the time interval between promoter activity exceeds the life span of the split GFP. For example, the HSN neurons in C. elegans express the egl-44 gene in the embryo (Wu et al., 2001, Genes Dev. 15: 789) and the cat-1 gene, which is needed for the late larval expression of serotonin (Desai et al., 1988, Nature 336: 638). HSN fluorescence was weak and rarely seen when RecGFP was generated from these promoters. Additionally, fewer cells than expected in adults fluoresced with Pₛₜₒ₋₆czgfp and P_{unc-4}nzgfp (FIG. 5B). Apparently, this expression was limited by the expression from the unc-4 promoter. More cells were seen with this combination, however, than with P_{unc-4}czgfp and P_{unc-4}nzgfp (FIG. 1D), presumably because of the increased formation of the reconstituted protein due to mass action from the production of CZGFP from the sto-6 promoter and possibly because of a greater stability of the reconstituted protein than of its parts. Although these results indicated that care should be used when expressing RecGFP, they also demonstrate that these constructs can be used to study temporal as well as spatial coexpression.

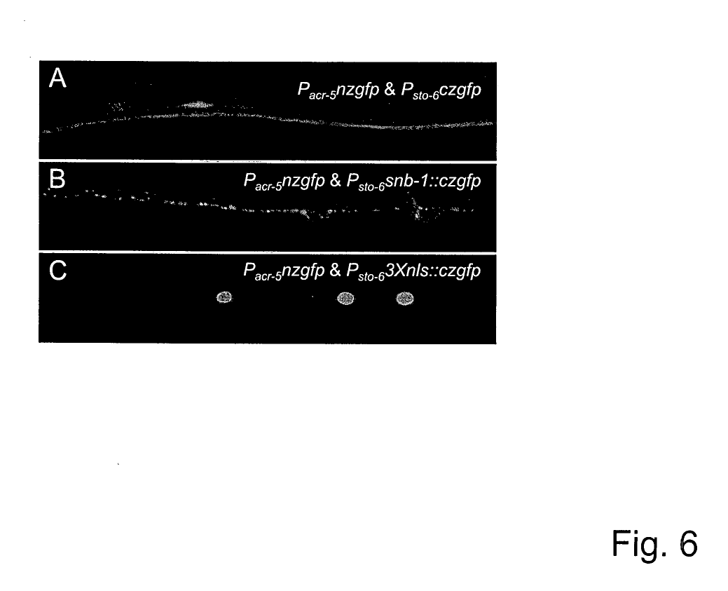

The combinatorial action of RecGFP can also be used to label cell constituents in a restricted set of cells. A synaptobrevin::GFP (SNB-1::GFP) protein fusion localizes to presynaptic vesicles (Nonet, 1999, J. Neurosci. Methods 89: 33). A split GFP version of this construct was produced. SNB-1 was fused with CZGFP and expressed from the sto-6 promoter. NZGFP was expressed from the acr-5 promoter. The resulting RecGFP fluorescence localized in the B motor neurons of the ventral cord cells in puncta (the presynaptic regions) (FIGS. 6A and B). The addition of SNB-1 caused the localization of the RecGFP in the cells. Fluorescence localized to nuclei, however, when CZGFP had a 3X nuclear localization signal (FIG. 6C).

Various patent and non-patent publications, including GenBank accession numbers, are cited herein, the contents of which are hereby incorporated by reference in their entireties.

We claim:

1. A method of detecting coordinate activity of a first and a second promoter element in a host cell containing a first nucleic acid comprising the first promoter operably linked to a nucleic acid encoding a first split fluorescent protein-construct and a second nucleic acid comprising the second promoter operably linked to a second nucleic acid encoding a second split fluorescent protein-construct, where the first and second split fluorescent protein-constructs are complementary and the first and second promoters are not the same, comprising detecting the formation of a reconstituted fluorescent protein from the split fluorescent protein-constructs by detecting fluorescence characteristic of the reconstituted fluorescent protein.

2. The method of claim 1, wherein the first and second split fluorescent protein-constructs each comprise a portion of the same parent fluorescent protein.

3. The method of claim 1, wherein the first and second split fluorescent protein-constructs each comprise a portion of a different parent fluorescent protein.

4. A method of marking a cell having a cell type of interest, comprising introducing, into the cell, a first nucleic acid comprising a first promoter operably linked to a nucleic acid encoding a first split fluorescent protein-construct and a second nucleic acid comprising a second promoter operably linked to a second nucleic acid encoding a second split fluorescent protein-construct, where the first and second split fluorescent protein-constructs are complementary and the first and second promoters are both active in the cell type of interest and are not the same.

5. The method of claim 4, wherein the first and second split fluorescent protein-constructs each comprise a portion of the same parent fluorescent protein.

6. The method of claim 4, wherein the first and second split fluorescent protein-constructs each comprise a portion of a different parent fluorescent protein.

7. The method of claim 4, wherein the cell is a member of a diverse cell population.

8. A method of marking a cell structure of interest, comprising introducing, into the cell, a first nucleic acid comprising a first promoter operably linked to a nucleic acid encoding a first split fluorescent protein-construct and a second nucleic acid comprising a second promoter operably linked to a second nucleic acid encoding a second split fluorescent protein-construct, where the first and second split fluorescent protein-constructs are complementary and the first and second promoters are both active in the cell type of interest, and one or both of the split fluorescent protein-constructs comprise a localization molecule that directs the split fluorescent protein-constructs to the cell structure of interest.

9. A method of determining whether a gene of interest is expressed in a specific cell type, comprising introducing, into a cell of the specific cell type, a first nucleic acid comprising the promoter of the gene of interest operably linked to a nucleic acid encoding a first split fluorescent protein-construct and a second nucleic acid comprising a second promoter operably linked to a second nucleic acid encoding a second split fluorescent protein-construct, where the first and second split fluorescent protein-constructs are complementary and the second promoter is active in the specific cell type, and detecting whether or not reconstituted fluorescent protein is produced, wherein the production of reconstituted fluorescent protein indicates that the gene of interest is expressed in the specific cell type.

10. The method of claim 9, wherein the first and second split fluorescent protein-constructs each comprise a portion of the same parent fluorescent protein.

11. The method of claim 9, wherein the first and second split fluorescent protein-constructs each comprise a portion of a different parent fluorescent protein.

12. A nucleic acid molecule comprising a promoter element operably linked to a nucleic acid encoding a split fluorescent protein-construct comprising a split fluorescent protein linked to a binder element and a localization molecule.

13. The nucleic acid molecule of claim 12, where the binder element does not comprise a leucine zipper.

14. A nucleic acid molecule comprising (i) a first nucleic acid encoding a first split fluorescent protein-construct, comprising a first promoter element operably linked to a nucleic acid encoding a first split fluorescent protein and a nucleic acid linked to a first binder element, and (ii) a second nucleic acid encoding a second split fluorescent protein-construct, comprising a second promoter element operably linked to a second nucleic acid encoding a second split fluorescent protein and a nucleic acid linked to a second binder element, wherein
the first and second split fluorescent proteins are complementary;
the first and second binder elements can form a bond selected from the group consisting of a non-covalent bond and a covalent bond; and
the first and second promoters are not the same.

15. A vector containing the nucleic acid molecule of claim 12.

16. A vector containing the nucleic acid molecule of claim 13.

17. A vector containing the nucleic acid molecule of claim 14.

18. A host cell containing the nucleic acid of claim 12.

19. A host cell containing the nucleic acid of claim 13

20. A host cell containing the nucleic acid of claim 14.

21. A host cell containing (i) a first nucleic acid encoding a first split fluorescent protein-construct, comprising a first promoter element operably linked to a nucleic acid encoding a first split fluorescent protein and a nucleic acid linked to a first binder element, and (ii) a second nucleic acid encoding a second split fluorescent protein-construct, comprising a second promoter element operably linked to a second nucleic acid encoding a second split fluorescent protein and a nucleic acid linked to a second binder element, wherein
the first and second split fluorescent proteins are complementary;
the first and second binder elements can form a bond selected from the group consisting of a non-covalent bond and a covalent bond; and
the first and second promoters are not the same.

22. A transgenic organism carrying, in its genome, a nucleic acid comprising a promoter element operably linked to a split fluorescent protein-construct.

23. The transgenic organism of claim 22, which is a unicellular organism.

24. The transgenic organism of claim 22, which is a multicellular organism.

25. The transgenic organism of claim 24, which is an embryonic organism.

26. The transgenic organism of claim 22, which is a plant.

27. The transgenic organism of claim 24 which is an animal selected from the group consisting of Caenorhabditis elegans, Drosophila melanogaster, Danio rerio, and Mus musculus.

28. A fluorescent protein having the sequence
(SEQ ID NO:1)
MSKGEELFTGVVPILVELDGDVNGHKFSVSGEGEGDATYGKLTLKFICTT
GKLPVPWPTLVTTFGYGLQCFARYPDHMKQHDFFKSAMPEGYVQERTIFF
KDDGNYKTRAEVKFEGDTLVNRIELKGIDFKEDGNILGHKLEYNYNSHNV
YIMADKQKNGIKANFKIRHNIEDGSVQLADHYQQNTPIGDGPVLLPDNHY
LSTQSALSKDPNEKRDHMVLLEFVTAAGITHGMDELYK.

29. A nucleic acid comprising a nucleic acid encoding the fluorescent protein of claim 46.


SEQUENCE LISTING (37 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 2 - DNA, 51 bp, Artificial Sequence
  synthetic oligonucleotide
gtaagtttaaacttggacttactaactaacggattatatttaaattttcag

SEQ ID NO: 3 - DNA, 51 bp, Artificial Sequence
  synthetic oligonucleotide
gtaagtttaaacatgattttactaactaactaatctgatttaaattttcag

SEQ ID NO: 4 - DNA, 32 bp, Artificial Sequence
  synthetic 5′ Primer
cgcggatccatggctagcaaaggagaagaact

SEQ ID NO: 5 - DNA, 32 bp, Artificial Sequence
  synthetic 3′ Primer
ccggaattctcactgagccagttctttcttca

SEQ ID NO: 6 - DNA, 28 bp, Artificial Sequence
  synthetic 5′ Primer
cgcggatccatggctagcgcacagctgg

SEQ ID NO: 7 - DNA, 33 bp, Artificial Sequence
  synthetic 3′ Primer
ccggaattctcagttgtacagttcatccatgcc

SEQ ID NO: 8 - DNA, 32 bp, Artificial Sequence; identical to SEQ ID NO: 4
  synthetic 5′ Primer

SEQ ID NO: 9 - DNA, 32 bp, Artificial Sequence
  synthetic 3′ Primer
ccggaattctcagccagagccagagccacctt

SEQ ID NO: 10 - DNA, 34 bp, Artificial Sequence
  synthetic 5′ Primer
gatcaagcttccccaaattggaacagtgaaatac

SEQ ID NO: 11 - DNA, 35 bp, Artificial Sequence
  synthetic 3′ Primer
gatcggatcccattttcactttttggaagaagaag

SEQ ID NO: 12 - DNA, 22 bp, Artificial Sequence
  synthetic 5′ Primer
catgtgattatgcatgcgaaag

SEQ ID NO: 13 - DNA, 25 bp, Artificial Sequence
  synthetic 3′ Primer
gcatgctgaaaattgtttttaaagc

SEQ ID NO: 14 - DNA, 29 bp, Artificial Sequence
  synthetic 5′ Primer
gtacaagcttgacaaaacaactttcttgg

SEQ ID NO: 15 - DNA, 37 bp, Artificial Sequence
  synthetic 3′ Primer
gtacggatccatttgatcctggaacatagataatttg

SEQ ID NO: 16 - DNA, 27 bp, Artificial Sequence
  synthetic 5′ Primer
tgaaataagcttcaattaattcgtcta

SEQ ID NO: 17 - DNA, 33 bp, Artificial Sequence
  synthetic 3′ Primer
cgcggatcccatgctcacaaccttcttggaagg

SEQ ID NO: 18 - DNA, 26 bp, Artificial Sequence
  synthetic 5′ Primer
aagcttgcatgcctgcagtaacattt

SEQ ID NO: 19 - DNA, 36 bp, Artificial Sequence
  synthetic 3′ Primer
cgcggatcccatagattgaatgtgtggtgcattcag

SEQ ID NO: 20 - DNA, 23 bp, Artificial Sequence
  synthetic 5′ Primer
cgcaagcttgaagctctcggaaa

SEQ ID NO: 21 - DNA, 33 bp, Artificial Sequence
  synthetic 3′ Primer
cgcggatcccattacactttgacttggatcacc

SEQ ID NO: 22 - DNA, 32 bp, Artificial Sequence
  synthetic 5′ Primer
cgcggatccataggagttccctctgacttcgc

SEQ ID NO: 23 - DNA, 36 bp, Artificial Sequence
  synthetic 3′ Primer
cgcggatcccataatctttgaaataagaactgggta

SEQ ID NO: 24 - DNA, 29 bp, Artificial Sequence
  synthetic 5′ Primer
acgcgtcgactggaccaccagcttgcagt

SEQ ID NO: 25 - DNA, 32 bp, Artificial Sequence
  synthetic 3′ Primer
cgcggatcccatgttttgtcggctcctaaaac

SEQ ID NO: 26 - DNA, 30 bp, Artificial Sequence
  synthetic 5′ Primer
cgcggatccgacgctcaaggagatgccggc

SEQ ID NO: 27 - DNA, 30 bp, Artificial Sequence
  synthetic 3′ Primer
cgcggatccttttcctccagcccataaaac

SEQ ID NO: 28 - DNA, 60 bp, Artificial Sequence
  synthetic 5′ Primer
ctataactcacacaatgtatacatcatggcagacaaacaaggtggctctggctctggcgc

SEQ ID NO: 29 - DNA, 57 bp, Artificial Sequence
  synthetic 3′ Primer
accggcgctcagttggaattctacgaatgctactgagccagttctttcttcagtgcc

SEQ ID NO: 30 - DNA, 56 bp, Artificial Sequence
  synthetic 5′ Primer
attttcaggaggacccttgagggtaccggtagaaaaaatggctagcgcacagctgg

SEQ ID NO: 31 - DNA, 59 bp, Artificial Sequence
  synthetic 3′ Primer
gtaaaatcatgtttaaacttacaactttgattccattcttaccgcttccaccctgtgcc

SEQ ID NO: 32 - DNA, 60 bp, Artificial Sequence
  synthetic 5′ Primer
ctatatttcacacaacgtatacatcactgccgacaaacaaggtggctctggctctggcgc

SEQ ID NO: 33 - DNA, 57 bp, Artificial Sequence; identical to SEQ ID NO: 29
  synthetic 3′ Primer

SEQ ID NO: 34 - DNA, 56 bp, Artificial Sequence; identical to SEQ ID NO: 30
  synthetic 5′ Primer

SEQ ID NO: 35 - DNA, 59 bp, Artificial Sequence
  synthetic 3′ Primer
gtaaaatcatgtttaaacttaccgctttgatcccattcttaccgcttccaccctgtgcc

SEQ ID NO: 36 - DNA, 57 bp, Artificial Sequence
  synthetic 5′ Primer
gcgggatccaccgccccaaagaagaaacgcaaagtaccgagctcagaaaaaatgacc

SEQ ID NO: 37 - DNA, 42 bp, Artificial Sequence
  synthetic 3′ Primer
gactggctagccattttttctaccggtactttgcgtttcttt